SEQUENCE LISTING (1 sequence)

SEQ ID NO: 1 - DNA, 1481 bp, Sphingomonas sp. KMK-001
agagtttgatcctggctcagaacgaacgctggcggcatgcctaatacatgcaagtcgaacgatcacttcggtggtagtggcgcacgggtgcgtaacgcgtgggaatctgcccttgggttcggaataacagttggaaacgactgctaataccggatgatgacgtaagtccaaagatttatcgcccaaggatgagcccgcgtaggattagctagttggtgaggtaaaggctcaccaaggcaacgatccttagctggtctgagaggatgatcagccacactgggactgagacacggcccagactcctacgggaggcagcagtagggaatattggacaatgggggcaaccctgatccagcaatgccgcgtgagtgatgaaggccttagggttgtaaagctcttttacccgagatgataatgacagtatcgggagaataagctccggctaactccgtgccagcagccgcggtaatacggagggagctagcgttgttcggaattactgggcgtaaagcgcacgtaggcggcgatttaagtcagaggtgaaagcccggggctcaaccccggaactgcctttgagactggattgctagaatcttggagaggcgggtggaattccgagtgtagaggtgaaattcgtagatattcggaagaacaccagtggcgaaggcggcccgctggacaagtattgacgctgaggtgcgaaagcgtggggagcaaacaggattagataccctggtagtccacgccgtaaacgatgataactagctgccggggcacatggtgtttcggtagcgcagctaacgcattaagttatccgcctggggagtacggtcgcaagattaaaactcaaaggaattgacgggggcctgcacaagcggtggagcatgtggtttaattcgaagcaacgcgcagaaccttaccaacgtttgacatccctatcgcggatcgtggagacactttccttcagttcggctggataggtgacaggtgctgcatggctgtcgtcagctcgtgtcgtgagatgttgggttaagtcccgcaacgagcgcaaccctcgcctttagttgccagcatttagttgggtactctaaaggaaccgccggtgataagccggaggaaggtggggatgacgtcaagtcctcatggcccttacgcgttgggctacacacgtgctacaatggcgactacagtgggcagccactccgcgaggaggagctaatctccaaaagtcgtctcagttcggattgttctctgcaactcaagagcatgaaggcggaatcgctagtaatcgcggatcagcatgccgcggtgaatacgttcccaggccttgtacacaccgcccgtcacaccatgggagttggattcacctgaaggcgctgcgctaactcgcaagagaggcaggcgaccacggtgggtttagcgactggggtgaagtcgtaacaaggtagccgtaggggaacctgcggctggatcacctcctt

(19) United States
(12) Patent Application Publication
(10) Pub. No.: US 2010/0215620 A1
(43) Pub. Date: Aug. 26, 2010

(54) THE CO-CULTURE METHOD OF SPHINGOMONAS SP. BACTERIAL STRAIN AND ASPERGILLUS SP. FUNGUS STRAIN, NEW ANTI-CANCER AND ANTIBIOTIC GLIONITRINS DERIVED FROM THIS CO-CULTURE METHOD, AND PHARMACEUTICAL COMPOSITION CONTAINING GLIONITRINS OR PHARMACEUTICALLY ACCEPTABLE SALT THEREOF AS AN ACTIVE INGREDIENT

(75) Inventors: Hyun Ok Yang, Gangwon-do (KR); Hak Cheol Kwon, Gangwon-do (KR); Hyun-Bong Park, Kyunggi-do (KR); Ji-Hye Yoo, Seoul (KR)

Correspondence Address: LUCAS & MERCANTI, LLP, 475 PARK AVENUE SOUTH, 15TH FLOOR, NEW YORK, NY, 10016, US

(73) Assignee: KOREA INSTITUTE OF SCIENCE AND TECHNOLOGY, Seoul (KR)

(21) Appl. No.: 12/738,597

(22) PCT Filed: Oct. 23, 2008
(86) PCT No.: PCT/KR08/06275

§ 371 Date: Apr. 16, 2010

(30) Foreign Application Priority Data
Nov. 20, 2007 (KR) 10-2007-0118544

(51) Int. Cl. A61K 35/74; C12N 1/20; C12N 1/14; C07F 15/00; C07D 241/36; C07D 285/15; C12P 17/16; C12P 17/18; C12P 13/00; A61K 31/555; A61K 31/498; A61K 31/548; A61P 31/04; A61P 35/00

(52) U.S. Cl. 424/93.4; 435/252.1; 435/256.1; 544/225; 544/344; 544/5; 435/118; 435/119; 435/128; 514/189; 514/250; 514/222.8

(57) ABSTRACT

The present invention relates to a co-culture method of Sphingomonas sp. bacterial strain and Aspergillus sp. fungus strain, in which the novel Sphingomonas sp. bacterial strain KMK-001 is cultured in a liquid medium and the novel Aspergillus sp. strain KMC-901 separately cultured in another liquid medium is added to the above culture solution, a novel glionitrin biosynthesized therefrom and a pharmaceutical composition comprising the said glionitrin or its pharmaceutically acceptable salt as an active ingredient. The glionitrin herein has strong cytotoxic effect on cancer cells and has antibiotic effect on 10 pathogenic bacteria including the novel Sphingomonas sp. bacterial strain KMK-001, so that it can be effectively applied in antibiotics or anti-cancer agents.

### TECHNICAL FIELD

The present invention relates to a co-culture method of a Sphingomonas sp. bacterial strain and an Aspergillus sp. fungus strain, in which the novel Sphingomonas sp. bacterial strain KMK-001 is cultured in a liquid medium and the novel Aspergillus sp. strain KMC-901 separately cultured in another liquid medium is added to the above culture solution, a novel glionitrin biosynthesized therefrom and a pharmaceutical composition comprising the said glionitrin or its pharmaceutically acceptable salt as an active ingredient.

### BACKGROUND ART

Studies have been undergoing to obtain a new material from natural sources. However, taking so much time, costs and labor for repeated separation processes from base materials is a major barrier for improving efficiency in the development of a new material having biological activity from the nature. One of important factors to develop a novel drug is to acquire a new material. “A new material development technique using co-culture” has been recently re-introduced as a modern art by Scripps Institution of Oceanography, USA, by which the development of new materials such as pestalone (Mercedes Cueto et al. J. Nat. Prod. 64: 1444-1446, 2001), libertellenone (Oh, D.-C. et al. Bioorg. Med. Chem. 13: 5267-5273, 2005), and emericellamide (Oh, D,-C. et al. J. Nat. Prod. 70: 515-520, 2007) has succeeded. Pestalone, libertellenone and emericellamide demonstrate strong anticancer- and anti-bacterial activity in vitro, providing possibility for the development of a novel anticancer agent and antibiotics. The co-culture method developed by Scripps Institution of Oceanography, USA, comprises the processes of adding a small amount of bacteria culture solution to fungi culture solution, which is though very different from the co-culture method of the present invention comprising the steps of adding a small amount of a fungi culture solution to a bacteria culture solution.

The present inventors developed a co-culture method of a Sphingomonas sp. bacterial strain and an Aspergillus sp. fungus strain for the first time and then completed this invention by confirming that the novel compound glionitrin produced by the method of the present invention had strong anticancer and antibacterial effect.

### DISCLOSURE

### Technical Problem

It is an object of the present invention to provide a novel Sphingomonas sp. bacterial strain, a novel Aspergillus sp. fungus strain, a co-culture method of a Sphingomonas sp. bacterial strain and an Aspergillus sp. fungus strain, a novel compound glionitrin produced by the above method and an anticancer agent or an antibacterial agent comprising glionitrin or its pharmaceutically acceptable salt as an active ingredient.

### Technical Solution

To achieve the above object, the present invention provides a Sphingomonas sp. bacterial strain deposited under the accession number KCCM 10888P.

The present invention also provides an Aspergillus sp. fungus strain deposited under the accession number KCCM 10889P.

The present invention also provides a co-culture method containing the step of culturing the bacterial mixture prepared by adding the Aspergillus sp. fungus strain separately cultured in a liquid medium or the culture solution thereof to the Sphingomonas sp. bacterial strain culture solution.

The present invention also provides a culture solution of the said bacterial mixture cultured by the co-culture method of the present invention.

The present invention also provides a compound represented by formula 1 or formula 2 separated from the culture solution of the bacterial mixture cultured by the co-culture method of the present invention.

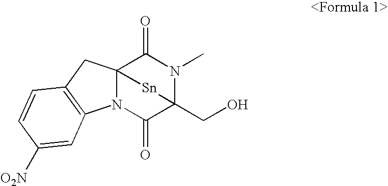

n is the number of S, which is 1-4; and

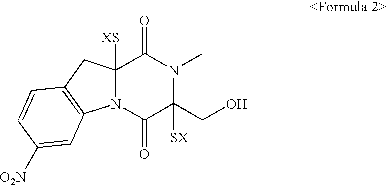

X is H or alkyl group, might be different or same, and contains isomers of asymmetric carbons.

The present invention also provides an anticancer agent containing the culture solution of the bacterial mixture cultured by the co-culture method of the present invention or the compound produced therefrom.

The present invention also provides an antibacterial agent containing the culture solution of the bacterial mixture cultured by the co-culture method of the present invention or the compound produced therefrom.

The present invention also provides a use of the said culture solution and the compound extracted therefrom for the production of an anticancer agent.

The present invention also provides a use of the said culture solution and the compound extracted therefrom for the production of an antibacterial agent.

The present invention also provides a health food comprising the culture solution and the compound separated therefrom for the prevention of cancer and improvement of health.

The present invention also provides a health food comprising the culture solution and the compound separated therefrom for the prevention of bacterial infection and improvement of health.

The present invention also provides a method for treating cancer containing the step of administering a therapeutically effective dose of the said culture solution and the compound extracted therefrom to a subject.

In addition, the present invention provides a method for treating bacterial infection containing the step of administering a therapeutically effective dose of the said culture solution and the compound extracted therefrom to a subject.

Hereinafter, the present invention is described in detail.

The present invention provides a Sphingomonas sp. bacterial strain KMK-001 deposited under the accession number KCCM10888P.

The present inventors collected water of pH 3.0 from the inside of Imgok Mine, Korea and performed centrifugation to give precipitate. The precipitate was suspended in saline to dilute it, followed by inoculation on a plate medium. During culture, single strain was separated and selected, which was then inoculated in a liquid medium, followed by culture. Chromosomal DNA of the obtained strain was separated and 16S rDNA sequencing was performed to identify the strain. As a result, the strain KMK-001 had 98.0% homology with Sphingomonas sp. A1XXyl1-5, suggesting that it was a novel strain of Sphingomonas. KMK-001 formed yellow mucous colonies in Capex-Dox medium and was confirmed to require nitrate and sulfate for growth, indicating that the strain was Gram-negative bacillus (see FIGS. 1 and 2). The strain KMK-001 was deposited at Korean Culture Center for Microorganisms (KCCM) on Nov. 7, 2007 (Accession No: KCCM10888P).

16S rDNA sequence of KMK-001, the novel strain of the present invention, is as follows:

(SEQ. ID. NO: 1)

AGAGTTTGAT CCTGGCTCAG AACGAACGCT GGCGGCATGC
CTAATACATG CAAGTCGAAC GATCACTTCG GTGGTAGTGG
CGCACGGGTG CGTAACGCGT GGGAATCTGC CCTTGGGTTC
GGAATAACAG TTGGAAACGA CTGCTAATAC CGGATGATGA
CGTAAGTCCA AAGATTTATC GCCCAAGGAT GAGCCCGCGT
AGGATTAGCT AGTTGGTGAG GTAAAGGCTC ACCAAGGCAA
CGATCCTTAG CTGGTCTGAG AGGATGATCA GCCACACTGG
GACTGAGACA CGGCCCAGAC TCCTACGGGA GGCAGCAGTA
GGGAATATTG GACAATGGGG GCAACCCTGA TCCAGCAATG
CCGCGTGAGT GATGAAGGCC TTAGGGTTGT AAAGCTCTTT
TACCCGAGAT GATAATGACA GTATCGGGAG AATAAGCTCC
GGCTAACTCC GTGCCAGCAG CCGCGGTAAT ACGGAGGGAG
CTAGCGTTGT TCGGAATTAC TGGGCGTAAA GCGCACGTAG
GCGGCGATTT AAGTCAGAGG TGAAAGCCCG GGGCTCAACC
CCGGAACTGC CTTTGAGACT GGATTGCTAG AATCTTGGAG
AGGCGGGTGG AATTCCGAGT GTAGAGGTGA AATTCGTAGA
TATTCGGAAG AACACCAGTG GCGAAGGCGG CCCGCTGGAC
AAGTATTGAC GCTGAGGTGC GAAAGCGTGG GGAGCAAACA
GGATTAGATA CCCTGGTAGT CCACGCCGTA AACGATGATA
ACTAGCTGCC GGGGCACATG GTGTTTCGGT AGCGCAGCTA
ACGCATTAAG TTATCCGCCT GGGGAGTACG GTCGCAAGAT
TAAAACTCAA AGGAATTGAC GGGGGCCTGC ACAAGCGGTG
GAGCATGTGG TTTAATTCGA AGCAACGCGC AGAACCTTAC
CAACGTTTGA CATCCCTATC GCGGATCGTG GAGACACTTT
CCTTCAGTTC GGCTGGATAG GTGACAGGTG CTGCATGGCT
GTCGTCAGCT CGTGTCGTGA GATGTTGGGT TAAGTCCCGC
AACGAGCGCA ACCCTCGCCT TTAGTTGCCA GCATTTAGTT
GGGTACTCTA AAGGAACCGC CGGTGATAAG CCGGAGGAAG
GTGGGGATGA CGTCAAGTCC TCATGGCCCT TACGCGTTGG
GCTACACACG TGCTACAATG GCGACTACAG TGGGCAGCCA
CTCCGCGAGG AGGAGCTAAT CTCCAAAAGT CGTCTCAGTT
CGGATTGTTC TCTGCAACTC AAGAGCATGA AGGCGGAATC
GCTAGTAATC GCGGATCAGC ATGCCGCGGT GAATACGTTC
CCAGGCCTTG TACACACCGC CCGTCACACC ATGGGAGTTG
GATTCACCTG AAGGCGCTGC GCTAACTCGC AAGAGAGGCA
GGCGACCACG GTGGGTTTAG CGACTGGGGT GAAGTCGTAA
CAAGGTAGCC GTAGGGGAAC CTGCGGCTGG ATCACCTCCT T

The present invention also provides an Aspergillus sp. fungus strain KMC-901 deposited under the accession number KCCM 10889P. The strain KMC-901 was deposited at Korean Culture Center for Microorganisms (KCCM) on Nov. 7, 2007 (Accession No: KCCM10889P).

The colony of KMC-901 looks like white wool at first and then the bluish green region in the center becomes larger over the time and at the same time, grey center region is formed instead, which is the same morphology as the colony of Aspergillus fumigatus (see FIG. 3) (Jeong Ga-Jin, Picture book to the microbiology, vol. 3: 192-194, 2007). Mycelium of KMC-901 has a septum and shows conidial head that is typical for Aspergillus sp. fungus (see FIG. 4) (Jeong Ga-Jin, Picture book to the microbiology, vol. 3: 192-194, 2007). The secondary metabolite of KMC-901 generated from liquid culture in Capex-Dox medium was analyzed by HPLC-MS and NMR. As a result, gliotoxin A and pseurotin A, specific components of Aspergillus fumigatus, were confirmed (Igarash, Y. et al. Journal of Antibiotic, 57: 748-754, 2004). From the morphological and chemical characteristics confirmed above, the strain KMC-901 was identified as an Aspergillus fumigatus sp. fungus and named as Aspergillus fumigatus KMC-901.

The present invention also provides a co-culture method containing the step of culturing the bacterial mixture prepared by adding the Aspergillus sp. fungus strain separately cultured in a liquid medium or the culture solution thereof to the Sphingomonas sp. bacterial strain culture solution.

In the co-culture method, the Sphingomonas sp. bacterial strain and the Aspergillus sp. fungus strain were mixed at the ratio of 1000:1.0˜1000:0.1 and more preferably at the ratio of 1000:0.5, but not always limited thereto.

The present inventors inoculated the novel bacterial strain KMC-001 and fungus strain KMC-901 in a liquid medium respectively, followed by culture. 500 μl of the fungus strain KMC-901 culture solution was added to 1 l of the bacterial strain KMK-001 culture solution, followed by co-culture. The co-culture solution was analyzed by HPLC to examine whether a new compound was produced. The bacterial strain KMK-001 and fungus strain KMC-901 were separately cultured for 15 days. Then the culture solution was also analyzed. As a result, glionitrin was not detected. In the meantime, when KMK-001 and KMC-901 were co-cultured for 15 days, glionitrin A was detected at the peak of retention time 18.072 and glionitrin B was detected at the peak of retention time 18.767. From the second day of the co-culture of KMK-001 and KMC-901, the co-culture solution was examined every other day. As a result, the production of glionitrin was confirmed from the 8^{th} day of the co-culture.

The present invention also provides a culture solution of the bacterial mixture cultured by the co-culture method of the present invention.

The present invention also provides a compound represented by formula 1 or formula 2 separated from the culture solution of the bacterial mixture cultured by the co-culture method of the present invention.

The novel compound herein is named as glionitrin by the present inventors.

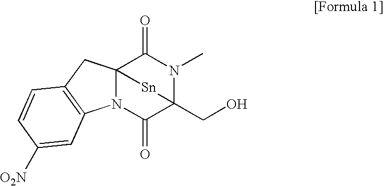

n is the number of S, which is 1-4.

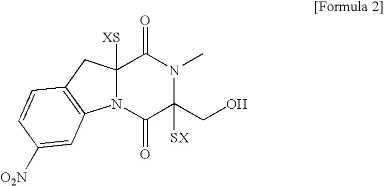

X is H or C₁-C₅ alkyl group, might be different or same, and contains isomers of asymmetric carbons.

The glionitrin of the present invention can be prepared by the method comprising the following steps:

1) co-culturing 1 l of the Sphingomonas sp. strain KMK-001 culture solution and 500 μl of the fungus strain KMC-901 culture solution, followed by extracting by adding an organic solvent or an absorption resin to the culture solution;

2) drying the extract of step 1) under reduced pressure, followed by obtaining fractions using column chromatography; and

3) separating and purifying the target compound from the fractions of step 2) using additional column chromatography.

In this method, the co-culture of step 1) is preferably performed for 10-20 days and more preferably for 12-18 days, but not always limited thereto. The organic solvent herein is ethyl acetate, butanol, methylene chloride or chloroform, but not always limited thereto. The extraction herein is performed by such methods using extraction devices as supercritical extraction, high pressure extraction or ultrasonic extraction or by those methods using absorption resins such as XAD and HP-20, but not always limited thereto. It is preferred to add an extraction solvent 1-3 times the volume of the co-culture solution and two times the volume is more preferred. Preferably, the extraction is performed at room temperature, but not always limited thereto. The extraction times are preferably 1-5 and more preferably 3, but not always limited thereto.

In this method, the drying under reduced pressure of step 2) is performed using rotary vacuum evaporator, but not always limited thereto. At this time, temperature for the drying under reduced pressure is preferably 20-40° C. and more preferably 30° C., but not always limited thereto.

In this method, the column chromatography of step 2) or step 3) is performed using a filler selected from the group consisting of silica gel, sephadex, RP18, polyamide, Toyopearl and XAD resin to separate and purify the target compound. The column chromatography can be repeated several times, if necessary, with properly selected fillers. At this time, a solvent is selected from the group consisting of chloroform(CHCl₃)-methanol, ethyl acetate-methanol, dichloromethane-methanol, methanol-water and acetonitrile-water, but not always limited thereto.

The ethyl acetate extract was dried under reduced pressure to give an extract, which proceeded to reversed phase column chromatography to give 6 fractions. Water and acetonitrile were used for elution. Precisely, elution was started with 20% acetonitrile/water and the content of acetonitrile was raised 20% every time. 100% methanol was used as the final solvent. The obtained fractions were analyzed by HPLC and as a result, the novel glionitrin was confirmed in 60% acetonitrile/water fraction. The fraction was purified by normal phase liquid chromatography using ethyl acetate and methylene chloride as solvents under isocratic condition of 90% methylene chloride/ethyl acetate and 60% methylene chloride/ethyl acetate. Glionitrin A represented by formula 3 was obtained from the fraction of retention time of 10 minutes and glionitrin B represented by formula 4 was obtained from the fraction of retention time of 15 minutes.

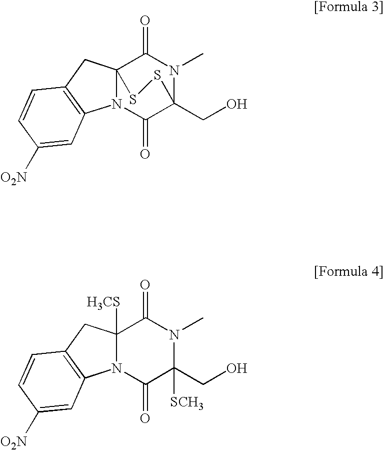

The chemical structures of the separated compounds were identified by using MS and nuclear spectrometer. As a result, it was confirmed that glionitrin A was amorphous light yellow powder represented by the molecular formula of C₁₃H₁₁N₃O₅S₂ which had the molecular weight of 353, and glionitrin B was colorless mucous semi-solid material represented by the molecular formula of C₁₅H₁₇N₃O₅S₂ which had the molecular weight of 383. When the glionitrin A was dissolved in water, acetonitrile aqueous solution, methanol aqueous solution or DMSO (dimethyl sulfoxide) solution at room temperature, trisulfide (glionitrin C) and tetrasulfide (glionitrin D) were generated and the chemical structures of them were also identified.

The present invention also provides an anticancer agent containing the culture solution of the bacterial mixture cultured by the co-culture method of the present invention or glionitrin.

Herein, the cancer is stomach cancer, liver cancer, colon cancer, lung cancer or prostatic cancer.

Cancer cell cytotoxicity of the glionitrin was investigated using AGS (stomach cancer, ATCC CRL-1739™), HepG2 (liver cancer, HB-8065™), HCT116 (colon cancer, CCL-247™), A549 (lung cancer, CCL-185™) and DU145 (prostatic cancer, HTB-81™) distributed from American Type Culture Collection (ATCC, Manassas, Va., USA).

The present inventors investigated cytotoxic effect of glionitrin on cancer cells. To do so, in vitro MTT (3-[4,5-dimethylthiazol-2-yl]-2,5-diphenyltrazdium bromide) assay was performed to quantify live cancer cells. As a result, glionitrin was confirmed to have strong anticancer effect on stomach cancer (AGS), liver cancer (HepG2), colon cancer (HCT-116), lung cancer (A549) and prostatic cancer (DU-145). Therefore, the culture solution of the bacterial mixture of the present invention or glionitrin can be effectively used as an anticancer agent.

The present invention also provides an antibacterial agent containing the culture solution of the bacterial mixture cultured by the co-culture method of the present invention or glionitrin.

Herein, the antibacterial agent has the antibacterial effect on Sphingomonas sp. bacterial strains.

The present inventors investigated antibacterial effect of glionitrin. Precisely, disc diffusion assay was performed to examine antibacterial effect of glionitrin on bacterial strains. Discs each absorbing glionitrin (experimental group and ampicillin (positive control group) were cultured on agar medium smeared with the bacterial strains, followed by observation. As a result, in the disc absorbing glionitrin, the bacteria were presented as yellow and the region indicating bacteria-non-growth was observed around the disc as colorless circle. This result indicates that glionitrin exhibited similar antibacterial effect to ampicillin used as the positive control.

The present inventors investigated antibacterial effect of glionitrin A and glionitrin B using Micrococcus leuteus IFC 12708, Bacillus subtilis ATCC 6633, Proteus vulgaris ATCC 3851, Salmonella typhimurium ATCC 1 4028 and 3 MRSA strains, Staphylococcus aureus ATCC 43300, S. aureus ATCC 700787 and S. aureus ATCC 700788 distributed from American Type Culture Collection (ATCC, Manassas, Va., USA). As a result, glionitrin B had no antibacterial effect on the every experimental group. In the meantime, glionitrin A demonstrated as strong antibacterial effect on 4 kinds of non-resistant bacteria as the positive control ampicillin. In particular, from the observation on three kinds of MRSA strains, glionitrin A demonstrated 15 times as strong antibacterial effect on the MSRA strains as the positive control ampicillin. Antifungal activity of glionitrin A and B on Aspergillus fumigatus HIC 6094 and Trichophyton rubrum IFO 9185 was also investigated. As a result, glionitrin B had no antifungal activity, while glionitrin A demonstrated antifungal activity similarly to the above.

Therefore, the culture solution of the bacterial mixture of the present invention or glionitrin can be effectively used as an antibacterial agent (see Table 3 and FIG. 6).

The anticancer agent or antibacterial agent of the present invention can contain a pharmaceutically acceptable salt in addition to the compound represented by formula 1 or formula 2. The pharmaceutically acceptable salt is preferably an acid addition salt prepared by using a pharmaceutically acceptable free acid. Whether it is inorganic or organic, a free acid can be used if it is pharmaceutically acceptable. Examples of the inorganic free acid are hydrochloric acid, hydrobromic acid, sulfuric acid, and phosphoric acid. Available organic free acids are exemplified by citric acid, acetic acid, lactic acid, tartaric acid, malic acid, fumaric acid, formic acid, propionic acid, oxalic acid, trifluoroacetic acid, benzoic acid, gluconic acid, methanesulfonic acid, glycolic acid, succinic acid, 4-toluenesulfonic acid, galacturonic acid, embonic acid, glutamic acid and aspartic acid. The anticancer agent or antibacterial agent of the present invention can contain not only a pharmaceutically acceptable salt but also any salt, hydrate and solvate prepared by the conventional method.

The anticancer agent or antibacterial agent of the present invention can selectively contain one or more compounds represented by formula 1 or formula 2 and additionally contain one or more active ingredients having the same or similar functions to the above components.

The anticancer agent or antibacterial agent of the present invention can be administered orally or parenterally and be used in general forms of pharmaceutical formulation. The anticancer agent or antibacterial agent of the present invention can be prepared for oral or parenteral administration by mixing with generally used diluents or excipients such as fillers, extenders, binders, wetting agents, disintegrating agents and surfactant. Solid formulations for oral administration are tablets, pills, powders, granules and capsules. These solid formulations are prepared by mixing the pharmaceutical composition of the present invention with one or more suitable excipients such as starch, calcium carbonate, sucrose or lactose, gelatin, etc. Except for the simple excipients, lubricants, for example magnesium stearate, talc, etc, can be used. Liquid formulations for oral administration are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations and suppositories. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerol and gelatin, etc. The anticancer agent or antibacterial agent of the present invention can be administered by parenterally and the parenteral administration includes subcutaneous injection, intravenous injection, and intramuscular injection.

The effective dosage of the anticancer agent or antibacterial agent of the present invention can be determined by those in the art according to weight and condition of a patient, severity of a disease, preparation of a drug, administration pathway and time.

The present invention also provides a use of the said culture solution and the compound extracted therefrom for the production of an anticancer agent or an antibacterial agent.

The culture solution of the bacterial mixture cultured by the co-culture method of the present invention or glionitrin can be effectively used for the production of an anticancer agent and an antibacterial agent. The anticancer agent or antibacterial agent of the present invention can contain not only a pharmaceutically acceptable salt but also any salt, hydrate and solvate prepared by the conventional method. The culture solution of the present invention and the compound separated therefrom can be used as a crucial component for the preparation of the said pharmaceutical composition. As explained hereinbefore, glionitrin has anticancer effect and antibacterial activity. So, it is well understood by those in the art that a compound produced by using the same has anticancer effect and antibacterial activity as well.

The present invention also provides a health food comprising the culture solution and the compound separated therefrom for the prevention of cancer or bacterial infection and improvement of health.

The culture solution of the bacterial mixture cultured by the co-culture method of the present invention or glionitrin can be used as food additive. In that case, the culture solution of the bacterial mixture cultured by the co-culture method of the present invention or glionitrin can be added as it is or as mixed with other food components according to the conventional method. The mixing ratio of active ingredients can be regulated according to the purpose of use (prevention, health enhancement or treatment). In general, to produce health food or beverages, the culture solution of the bacterial mixture cultured by the co-culture method of the present invention or glionitrin is added preferably by 0.1-15 weight part and more preferably by 0.1-10 weight part. However, if long term administration is required for health and hygiene or regulating health condition, the content can be lower than the above but higher content can be accepted as well since the culture solution of the bacterial mixture cultured by the co-culture method of the present invention or glionitrin has been proved to be very safe.

The food herein is not limited. For example, the culture solution of the bacterial mixture cultured by the co-culture method of the present invention or glionitrin can be added to meats, sausages, breads, chocolates, candies, snacks, cookies, pizza, ramyuns, flour products, gums, dairy products including ice cream, soups, beverages, tea, drinks, alcohol drinks and vitamin complex, etc, and in wide sense, almost every food applicable in the production of health food can be included.

The composition for health beverages of the present invention can additionally include various flavors or natural carbohydrates, etc, like other beverages. The natural carbohydrates above can be one of monosaccharides such as glucose and fructose, disaccharides such as maltose and sucrose, polysaccharides such as dextrin and cyclodextrin, and glucose alcohols such as xilytole, sorbitol and erythritol. Besides, natural sweetening agents such as thaumatin and stevia extract, and synthetic sweetening agents such as saccharin and aspartame can be included as a sweetening agent. The content of the natural carbohydrate is preferably 0.01-0.04 g and more preferably 0.02-0.03 g in 100 ml of the culture solution of the bacterial mixture cultured by the co-culture method of the present invention or glionitrin.

In addition to the ingredients mentioned above, the culture solution of the bacterial mixture cultured by the co-culture method of the present invention or glionitrin can include in variety of nutrients, vitamins, minerals, flavors, coloring agents, pectic acid and its salts, alginic acid and its salts, organic acid, protective colloidal viscosifiers, pH regulators, stabilizers, antiseptics, glycerin, alcohols, carbonators which used to be added to soda, etc. The culture solution of the bacterial mixture cultured by the co-culture method of the present invention or glionitrin can also include natural fruit juice, fruit beverages and/or fruit flesh addable to vegetable beverages. All the mentioned ingredients can be added singly or together. The mixing ratio of those ingredients does not matter in fact, but in general, each can be added by 0.01-1 weight part per 100 weight part of the culture solution of the bacterial mixture cultured by the co-culture method of the present invention or glionitrin.

In addition, the present invention provides a method for treating cancer or bacterial infection containing the step of administering a therapeutically effective dose of the said culture solution and the compound extracted therefrom to a subject.

Herein, the subject is preferably human or any other mammals. The mammals herein can be selected from the group consisting of mouse, rat, guinea pig, pig, rabbit, monkey, and chimpanzee, but not always limited thereto.

The culture solution and the compound extracted therefrom can be administered orally or parenterally (for example, intravenous, hypodermic, local or peritoneal injection). The effective dosage of the culture solution and the compound extracted therefrom can be determined according to weight, age, gender, health condition, diet, administration frequency, administration method, excretion and severity of a disease. The dosage unit can contain, for example, 1, 2, 3 or 4 individual doses or ½, ⅓ or ¼ of an individual dose. An individual dose preferably contains the amount of active compound which is administered in one application and which usually corresponds to a whole, ½, ⅓ or ¼ of a daily dose.

### ADVANTAGEOUS EFFECT

The novel co-culture method of Sphingomonas sp. bacteria-Aspergillus sp. fungi is expected to be a promising technique to obtain a new material. The novel compound produced by the said co-culture method, glionitrin, has strong cytotoxic effect on cancer cells and antibacterial effect on 10 kinds of pathogenic bacteria including KMK-001. Therefore, it can be effectively used for the development of an anticancer agent or antibiotics.

### DESCRIPTION OF DRAWINGS

The application of the preferred embodiments of the present invention is best understood with reference to the accompanying drawings, wherein:

FIG. 1 is a photograph illustrating the morphology of the novel Sphingomonas sp. bacterial strain on CDA medium,

FIG. 2 is a microphotograph illustrating the Gram-stained strain,

FIG. 3 is a photograph illustrating the morphology of the Aspergillus fumigatus fungus strain, KMC-901, on CDA medium,

FIG. 4 is a microphotograph illustrating the mycelium having the septum and the ascus of conidiophore of the strain,

FIG. 5 is a microphotograph illustrating the strains in the co-culture solution of the bacterial strain KMK-001 and the fungus strain KMC-901,

FIG. 6 is a photograph illustrating the antibacterial activity of glionitrin on the said strains:

Left: disc absorbed with ampicillin; and

Right: disc absorbed with glionitrin,

FIG. 7 is a diagram illustrating the chromatogram of the single culture solution in which the bacterial strain KMK-001 was cultured for 15 days,

FIG. 8 is a diagram illustrating the chromatogram of the single culture solution in which the fungus strain KMC-901 was cultured for 15 days,

FIG. 9 is a diagram illustrating the chromatogram of the culture solution in which KMK-001 and KMC-901 were co-cultured for 15 days,

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Screening and Selection of Bacterial and Fungus Strains for Co-Culture

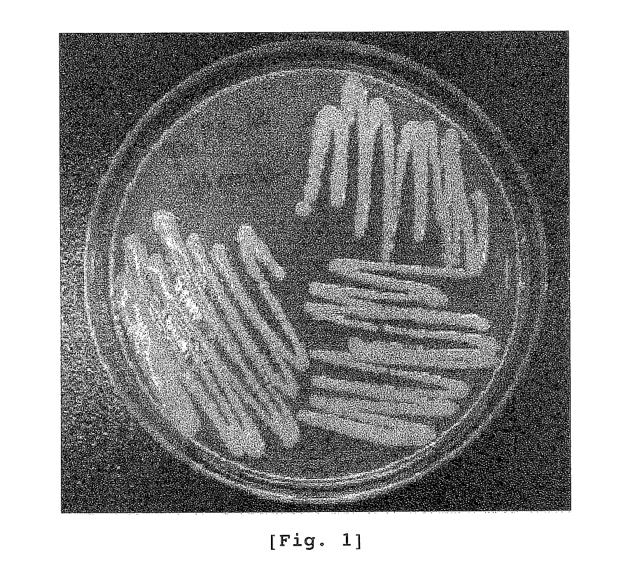

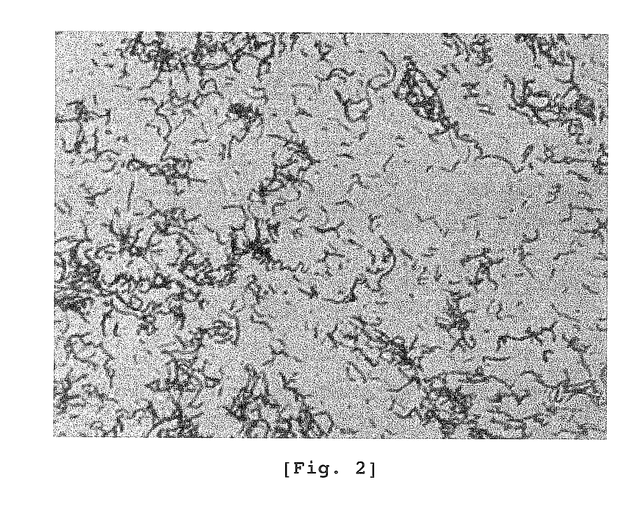

The present inventors collected water of pH 3.0 from the inside of Imgok Mine, Gangneung-si, Gangwondo, Korea and performed centrifugation to give precipitate. The precipitate was suspended and diluted in saline and the diluted precipitate was inoculated on YM agar medium (YMA) and Capex-Dox agar medium (CDA), followed by culture at 25° C. for 10 days. Single strain was separated and thereby 300 strains were obtained. Among them, bacterial and fungus strains to be used for co-culture were selected, which were then inoculated in Capex-Dox liquid medium, followed by culture in a 25° C. shaking incubator for 5-7 days. Chromosomal DNA of the obtained strain was separated and 16S rDNA sequencing was performed to identify the strain. As a result, the strain KMK-001 had 98.0% homology with Sphingomonas sp. A1XXyl1-5, suggesting that it was a novel strain of Sphingomonas. KMK-001 formed yellow mucous colonies in Capex-Dox medium (30 g saccharose, 3 g sodium nitrate, 1 g dipotassium phosphate, 0.5 g magnesium sulfate, 0.5 g potassium chloride, 0.01 g, ferrous sulfate in 1 L of DI water) and was confirmed to require nitrate and sulfate for growth, indicating that the strain was Gram-negative bacillus (see FIGS. 1 and 2). The strain KMK-001 was deposited at Korean Culture Center for Microorganisms (KCCM) on Nov. 7, 2007 (Accession No: KCCM10888P).

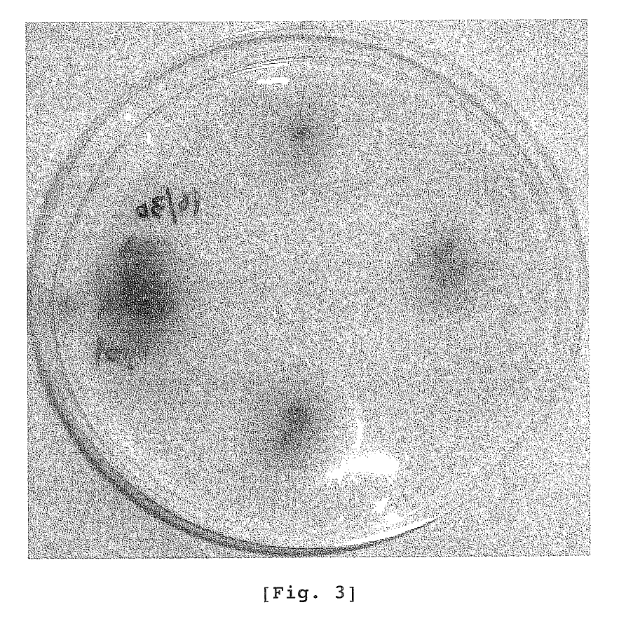

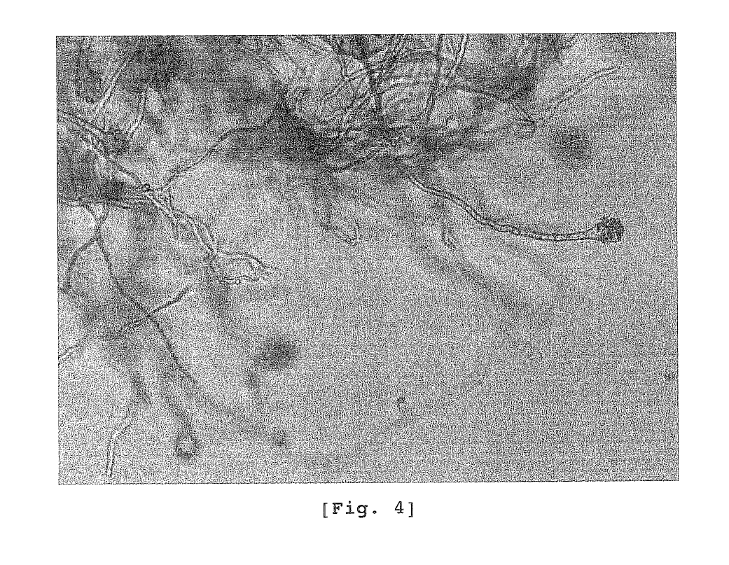

The present inventors also collected suspension of water and coal from the underground tunnel (depth of 150 m) of Jangseong Mine, Taebak-si, Gangwon-do, Korea, followed by centrifugation to give precipitate. The precipitate was suspended and diluted in sterilized purified water and the diluted precipitate was inoculated on Capex-Dox agar medium (CDA), followed by culture at 25° C. for 14 days. Single fungus strain was separated and the fungus strain KMC-901 was obtained therefrom. KMC-901, separated as a single strain, was cultured again on Capex-Dox agar medium (CDA), and then growth and color were observed. Mycelium, hypha, ceptum, conidiophore and ascus were also observed to identify the species. The compound generated by KMC-901 proceeded to chemical analysis and the result was used for identification of the strain. The colony of KMC-901 was white wool shape in the early stage but later the bluish green center region became larger and darker and a grey center was formed instead, which was consistent with the morphology of Aspergillus fumigatus (see FIG. 3). Mycelium of KMC-901 had septum and conidial head which is typical in Aspergillus sp. fungus (see FIG. 4). The secondary metabolite of KMC-901 generated from liquid culture in Capex-Dox medium was analyzed by HPLC-MS and NMR. As a result, gliotoxin A and pseurotin A, specific components of Aspergillus fumigatus, were confirmed. From the morphological and chemical characteristics confirmed above, the strain KMC-901 was identified as an Aspergillus fumigatus sp. Fungus strain and named as Aspergillus fumigatus KMC-901. The strain KMC-901 was deposited at Korean Culture Center for Microorganisms (KCCM) on Nov. 7, 2007 (Accession No: KCCM10889P).

### Example 2

### Co-Culture of the Novel Sphingomonas Sp. KMK-001 and Aspergillus Sp. Strain KMC-901

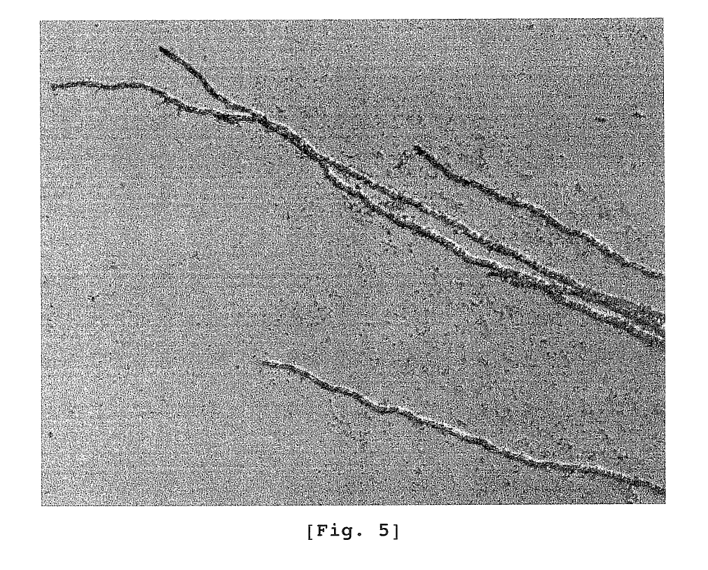

The present inventors inoculated the novel bacterial strain KMK-001 and fungus strain KMC-901 respectively in Capex-Dox liquid medium, followed by culture in a 25° C. shaking incubator for 3 days. The said two strains were cultured in 1 l Erlenmeyer flasks containing 0.5 l of Capex-Dox liquid medium for mass-production. 2 days later, 250 μl of the fungus strain KMC-901 culture solution was added to the Sphingomonas sp. strain KMK-001 culture solution, followed by co-culture. The co-culture solution containing those strains was observed under microscope. As shown in FIG. 5, bacterial strain was shown along with the fungus strain around the mycelium (see FIG. 5).

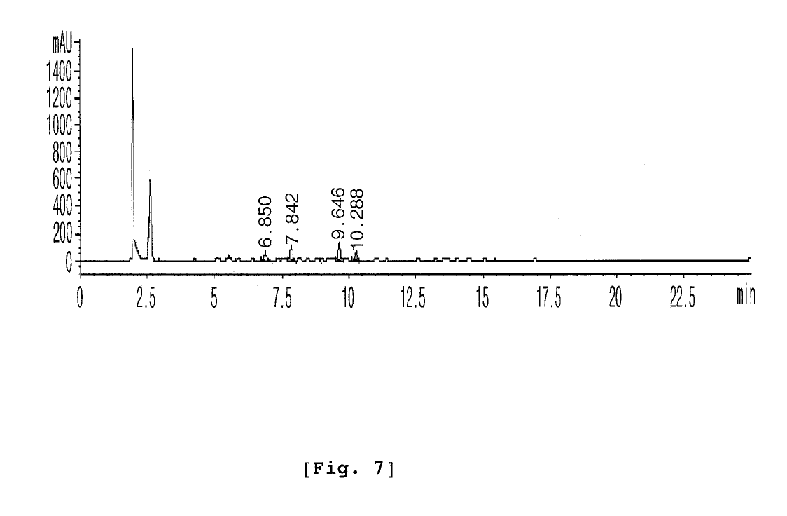

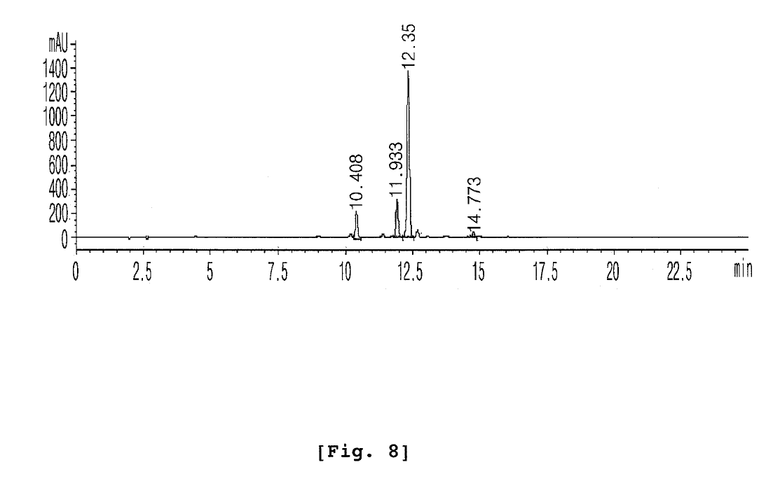

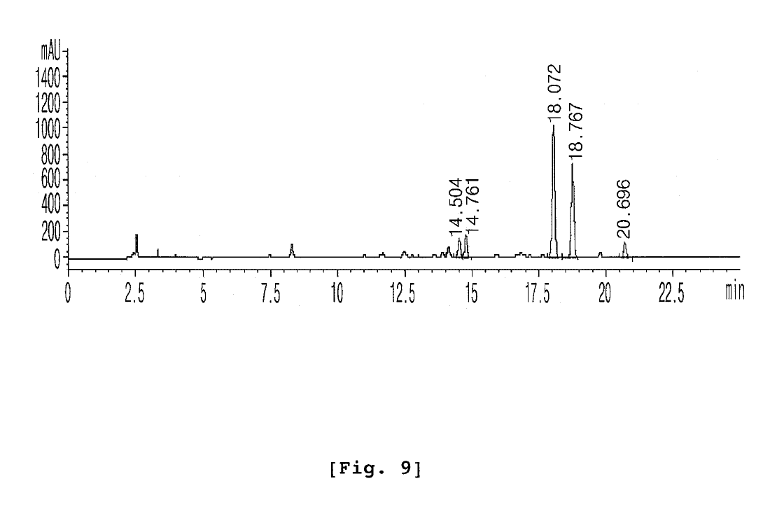

Production of a new compound in the co-culture solution was investigated by HPLC. HPLC was performed under the following conditions; [device: Agilent 1100 LC/MS system; elution speed: 0.7 mL/min; eluent: acetonitrile content was raised from 10% acetonitrile/water to 100% acetonitrile for 30 minutes; column: Phenomenex Luna 5 u C18(2) 4.6×150 mm; detector: DAD UV detector (254 nm)]. As shown in FIGS. 7-9, glionitrin was not detected in the culture solution wherein the bacterial strain KMK-001 or the fungus strain KMC-901 was single-cultured for 15 days. In the meantime, in the co-culture solution wherein the bacterial strain KMK-001 and the fungus strain KMC-901 were co-cultured for 15 days, glionitrin A was detected at the peak of retention time 18.072 and glionitrin B was detected at the peak of retention time 18.767. From the second day of the co-culture of KMK-001 and KMC-901, the co-culture solution was examined every other day. As a result, the production of glionitrin was confirmed from the 8^{th} day of the co-culture.

### Example 3

### Separation and Purification of Glionitrin

The present inventors extracted organic compounds from the culture solution obtained in Example 2 using ethyl acetate. The extract was dried under reduced pressure at 30° C. using a rotary vacuum evaporator and the resultant extract proceeded to reversed phase column chromatography, and as a result, 6 fractions were obtained. Water and acetonitrile were used for elution. Precisely, elution was started with 20% acetonitrile/water and the content of acetonitrile was raised 20% every time. 100% methanol was used as the final solvent. The obtained fractions were analyzed by HPLC and as a result, the novel glionitrin was confirmed in 60% acetonitrile/water fraction. The fraction was purified by normal phase liquid chromatography using ethyl acetate and methylene chloride as solvents under isocratic condition of 90% methylene chloride/ethyl acetate and 60% methylene chloride/ethyl acetate. Glionitrin A was obtained from the fraction of retention time of 10 minutes and glionitrin B was obtained from the fraction of retention time of 15 minutes.

### Example 4

### Instrumental Analysis of Glionitrin

The present inventors performed instrumental analysis with glionitrins to determine the chemical formulas of glionitrin A and glionitrin B. The separated glionitrin A was left in water or DMAO at room temperature. Then, trisulfide (glionitrin C) and tetrasulfide (glionitrin D) were generated therein and their chemical structures were identified by MS and NMR. ¹H and ¹³C NMR spectrums were observed at 500 MHz and 125 MHz respectively. Glionitrin A was measured in chloroform-d solvent and glionitrin B was measured in methanol-d₄ solvent.

TABLE 1
Chemical shift values of ¹H and ¹³C NMR of structures of glionitrins A and B

| Glionitrin A | | | | | Glionitrin B | | | | |
|---|---|---|---|---|---|---|---|---|---|
| Site | δ_{H} | Mult(J Hz) | δc | | Site | δ_{H} | Mult(J Hz) | δc | |
| 1 | | | 165.5 | C | 1 | | | 166.1 | C |
| 2-NMe | 3.30 | s | 27.8 | CH₃ | 2-NMe | 3.20 | s | 28.1 | CH₃ |
| | | | | | 3-SMe | 2.36 | s | 12.2 | CH₃ |
| 3 | | | 76.7 | C | 3 | | | 72.4 | C |
| 3aα | 4.34 | dd(12.5, 10.0) | 60.9 | CH₂ | 3aA | 4.39 | d(12.0) | 63.9 | CH₂ |
| 3aβ | 4.52 | dd(12.5, 6.0) | | | 3aB | 3.98 | d(12.0) | | |
| 3a-OH | 3.42 | dd(10.0, 6.0) | | | 3a-OH | | | | |
| 4 | | | 161.4 | C | 4 | | | 163.0 | C |
| 5-N | | | | | 5-N | | | | |
| 5a | | | 139.2 | C | 5a | | | 141.9 | C |
| 6 | 8.75 | d(2.5) | 111.4 | CH | 6 | 8.83 | d (2.5) | 112.2 | CH |
| 7 | | | 148.9 | C | 7 | | | 148.0 | C |
| 8 | 8.14 | br dd(8.5, 2.5) | 121.7 | CH | 8 | 8.14 | br dd(8.5, 2.5) | 121.2 | CH |
| 9 | 7.53 | br d(8.5) | 126.0 | CH | 9 | 7.62 | br d(8.5) | 125.9 | CH |
| 9a | | | 135.3 | C | 9a | | | 137.3 | C |
| 10α | 3.44 | d(19.0) | 36.8 | CH₂ | 10A | 3.73 | br s | 39.2 | CH₂ |
| 10β | 4.40 | br dd(19.0, 1.0) | | | 10B | | | | |
| | | | | | 10a-SMe | 2.27 | s | 13.2 | CH₃ |
| 10a | | | 74.4 | C | 10a | | | 71.8 | C |

<Glionitrin C>

ESI-MS: m/z 386 [M+H]⁺;

¹H NMR (500 MHz, CDCl₃): 3.40 (3H, s), 3.46 (1H, dd, J=19.0, 1.0 Hz), 4.29 (1H, dd, J=19, 1.0 Hz), 4.42 (1H, br d, J=12.5 Hz), 4.80 (1H, br d, J=12.5 Hz), 7.44 (1H, br d, J=8.5 Hz), 8.10 (1H, dd, J=8.5, 2.0 Hz), 8.89 (1H, d, J=2.0 Hz).

<Glionitrin D>

ESI-MS: m/z 440 [M+Na]⁺;

¹H NMR (500 MHz, CDCl₃): 3.21 (3H, s), 3.48 (1H, br dd, J=18.5, 1.0 Hz), 4.02 (1H, br dd, J=18.5, 1.0 Hz), 4.12 (1H, br d, J=12.5 Hz), 4.40 (1H, br d, J=12.5 Hz), 7.52 (1H, br d, J=8.5 Hz), 8.18 (1H, dd, J=8.5, 2.0 Hz), 9.18 (1H, d, J=2.0 Hz).

### Example 5

### Cytotoxic Effect of Glionitrin on Cancer Cells and Antibacterial Activity of Glionitrin Against the Bacterial Strain KMK-001

The present inventors investigated cytotoxic effect of glionitrin on cancer cells with in vitro MTT (3-[4,5-dimethylthiazol-2-yl]-2,5-diphenyltrazdium bromide) assay to quantify live cancer cells. Cancer cell cytotoxicity of glionitrin was evaluated using AGS (stomach cancer, ATCC CRL-1739™), HepG2 (liver cancer, HB-8065™), HCT116 (colon cancer, CCL-247™), A549 (lung cancer, CCL-185™) and DU145 (prostatic cancer, HTB-81™) distributed from American Type Culture Collection (ATCC, Manassas, Va., USA). Stomach cancer (AGS), liver cancer (HepG2), colon cancer (HCT-116), lung cancer (A549) or prostatic cancer (DU-145) cells were distributed in a 96-well plate at the density of 1×10⁴ cells/100 μl/well, followed by culture in a 37° C., CO₂ incubator for 24 hours. Then, the medium was replaced with a fresh one containing glionitrin, followed by culture at 37° C. 24 hours later, MTT solution was added thereto by 10 μl/well, followed by further culture for 1 hour at 37° C. One hour later, formazane generated by reductase in live cells was quantified by measuring OD₄₅₀.

TABLE 2
In vitro cytotoxic effect of glionitrins
A and B on 6 cancer cell lines

IC₅₀ (mM)

| Cancer cell line | glionitrin A | glionitrin B |
|---|---|---|
| AGS | 0.45 ± 0.11 | 1.25 ± 0.2 |
| DU-145 | 0.24 ± 0.02 | 2.29 ± 0.14 |
| HCT-116 | 0.82 ± 0.11 | 3.28 ± 0.55 |
| A549 | 0.55 ± 0.07 | 2.25 ± 0.25 |
| HepG2 | 2.28 ± 0.3 | >2 uM |
| MCF-7 | >2 uM | >2 uM |

As shown in Table 2, glionitrins A and B had strong cytotoxic effect on the said cancer cell lines.

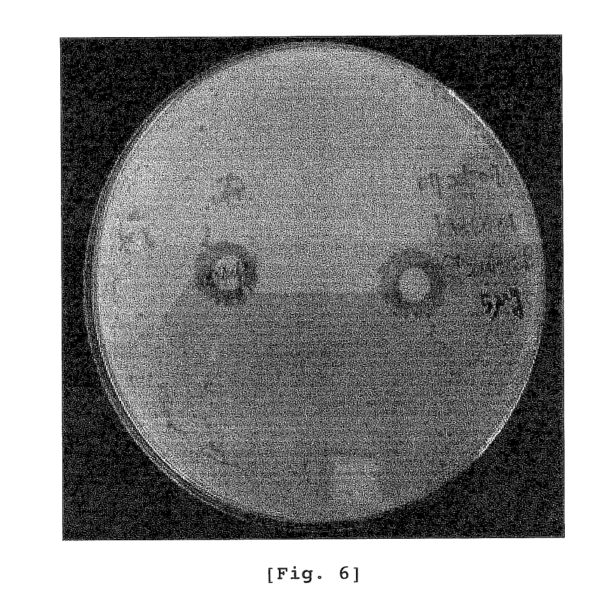

The present inventors also investigated antibacterial activity of glionitrins A and B against KMK-001 by disc diffusion assay. KMK-001 was smeared on Capex-Dox agar medium, on which the sterilized disc absorbing glionitrin was placed, followed by culture at 25° C. for 5 days. For the positive control, the sterilized disc absorbing ampicillin instead of glionitrin was placed and cultured under the same conditions. As a result, glionitrin was confirmed to have antibacterial activity similar to that of ampicillin (see FIG. 6).

The present inventors also investigated antibacterial effect of glionitrin A and glionitrin B using Micrococcus leuteus IFC 12708, Bacillus subtilis ATCC 6633, Proteus vulgaris ATCC 3851, Salmonella typhimurium ATCC 1 4028 and 3 MRSA strains, Staphylococcus aureus ATCC 43300, S. aureus ATCC 700787 and S. aureus ATCC 700788 distributed from American Type Culture Collection (ATCC, Manassas, Va., USA). Glionitrin B had no antibacterial activity at all in every experimental group. On the contrary, glionitrin A demonstrated as strong antibacterial activity as ampicillin (the positive control) against 4 non-resistant strains (MIC: 0.78-3.13 ug/mL). In particular, glionitrin A demonstrated 15 times as high antibacterial activity against 3 MRSA strains as ampicillin used as the positive control (MIC: 0.78 ug/ml) (see Table 3). To determine MIC of glionitrin for the said 7 bacterial strains, those 7 bacterial strains were cultured in standard methods broth (Difco) for 24 hours. Then, the medium was eliminated and 100 μl of medium containing 10⁵ cfu/mL of each strain was loaded in each well of a 96-well plate. Glionitrin diluted at different concentrations was added thereto, followed by culture at 37° C. for 24 hours. 24 hours later, OD₆₀₀ was measured.

The present inventors also evaluated antifungal activity of glionitrins A and B against those strains distributed from American Type Culture Collection (ATCC, Manassas, Va., USA) such as Aspergillus fumigatus HIC 6094 and Trichophyton rubrum IFO 9185. As a result, glionitrin B did not show antifungal activity, which was consistent with the above result. In the meantime, glionitrin A demonstrated antifungal activity (MIC: 12.5 ug/mL) (see Table 3). To determine MIC of glionitrin for those two fungus strains, spores of the fungus strains were suspended in sterilized distilled water at the density of 10⁵ spores/mL and the density was adjusted by using Sabouraud glucose liquid medium (Difco) to 10³ cells/mL. Then, the cells were distributed in a 96-well plate at the density of 10² cells/100 μl/well, to which glionitrin was added at different concentrations, followed by culture at 28° C. for hours. The concentration of glionitrin at which no fungi growth was confirmed was determined as MIC.

TABLE 3
In vitro antibacterial activity of glionitrins
A and B against 9 pathogenic microorganisms

| | | | Antibacterial | Antifungal |
|---|---|---|---|---|
| | | | control | control |
| | glionitrin | glionitrin | ampicillin | amphoteric |
| Pathogenic | AMIC | BMIC | MIC | in B MIC |
| microorganism | (ug/mL) | (ug/mL) | (ug/mL) | (ug/mL) |
| Micrococus | 0.78 | >100 | 0.78 | — |
| leuteus IFC | | | | |
| 12708[G(+)] | | | | |
| Bacillus subtilis | 6.25 | >100 | 3.13 | — |
| ATCC 6633 [G(+)] | | | | |
| Proteus vulgaris | 3.13 | >50 | 1.56 | — |
| ATCC 3851[G(−)] | | | | |
| Salmonella | 3.13 | >50 | 12.5 | — |
| typhimurium ATCC | | | | |
| 14028[G(−)] | | | | |
| Staphylococcu | 0.78 | >50 | 12.5 | — |
| aureus ATCC | | | | |
| 4330(MRSA) | | | | |
| S. aureus ATCC | 0.78 | >50 | 12.5 | — |
| 700787(MRSA) | | | | |
| S. aureus ATCC | 0.78 | >50 | 25.0 | — |
| 700788(MRSA) | | | | |
| Aspergillus | 12.5 | >50 | — | 1.56 |
| fumigatus HIC | | | | |
| 6094 | | | | |
| Trichophyton | 12.5 | >50 | — | 1.56 |
| rubrum IFO 9185 | | | | |

AS shown in Table 3, glionitrin A demonstrated strong antibacterial activity against those pathogenic microorganisms.

The Manufacturing Examples of the composition for the present invention are described hereinafter.

### Manufacturing Example 1

### Preparation of Powders

| | |
|---|---|
| Compound of formula 3 or formula 4 | 20 mg |
| Lactose | 100 mg |
| Talc | 10 mg |

Powders were prepared by mixing all the above components, which were filled in airtight packs according to the conventional method for preparing powders.

### Manufacturing Example 2

### Preparation of Tablets

| | | |
|---|---|---|
| Compound of formula 3 or formula 4 | 10 | mg |
| Corn starch | 100 | mg |
| Lactose | 100 | mg |
| Maqnesium stearate | 2 | mg |

Tablets were prepared by mixing all the above components by the conventional method for preparing tablets.

### Manufacturing Example 3

### Preparation of Pharmaceutical Capsules

| | | |
|---|---|---|
| Compound of formula 3 or formula 4 | 10 | mg |
| Crystalline cellulose | 3 | mg |
| Lactose | 14.8 | mg |
| Magnesium stearate | 0.2 | mg |

Capsules were prepared by mixing all the above components, which were filled in gelatin capsules according to the conventional method for preparing capsules.

### Manufacturing Example 4

### Preparation of Injections

| | | |
|---|---|---|
| Compound of formula 3 or formula 4 | 10 | mg |
| Mannitol | 180 | mg |
| Sterilized distilled water for injection | 2974 | mg |
| Na₂HPO₄, 12H₂O | 26 | mg |

Injections were prepared by mixing all the above components according to the conventional method for preparing injections (2 ml/ampoule).

### Manufacturing Example 5

### Preparation of Solutions

| | | |
|---|---|---|
| Compound of formula 3 or formula 4 | 20 | mg |
| Isomerized sugar | 10 | g |
| Mannitol | 5 | g |

| | |
|---|---|
| Purified water | proper amount |

Solutions were prepared by mixing all the above components according to the conventional method for preparing solutions. Particularly, all the above components were mixed, to which proper amount of lemon flavor and purified water were added to make 100 ml of solution. The solution was brown bottles and sterilized.

### INDUSTRIAL APPLICABILITY

The novel co-culture method of Sphingomonas sp. bacteria-Aspergillus sp. fungi is expected to be a promising technique to obtain a new material. The novel compound produced by the said co-culture method, glionitrin, has strong cytotoxic effect on cancer cells and antibacterial effect on 10 kinds of pathogenic bacteria including KMK-001. Therefore, it can be effectively used for the development of an anticancer agent or antibiotics.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A Sphingomonas sp. bacterial strain deposited under the accession number KCCM10888P.

2. An Aspergillus sp. fungus strain deposited under the accession number KCCM10889P.

3. A co-culture method containing the step of culturing the bacterial mixture prepared by adding the Aspergillus sp. fungus strain of claim 2 separately cultured in a liquid medium or the culture solution thereof to the culture solution of the Sphingomonas sp. bacterial strain of claim 1.

4. The co-culture method according to claim 3, wherein the Sphingomonas sp. bacterial strain is mixed with the Aspergillus sp. fungus strain at the ratio of 1000:1.0-1000:0.1.

5. A culture solution of the bacterial mixture cultured by the co-culture method of claim 3.

6. A compound represented by formula 1 or formula 2, separated from the culture solution of the bacterial mixture cultured by the co-culture method of claim 3: 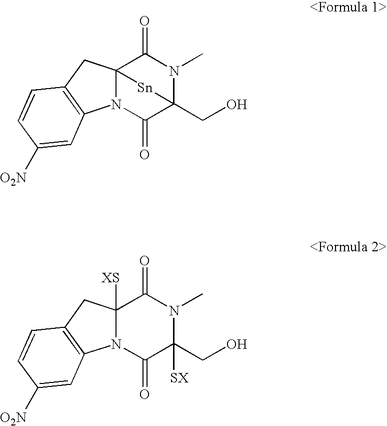
wherein
n is the number of S, which is 1-4; and
X is H or alkyl group, might be different or same, and contains isomers of asymmetric carbons.

7. The compound according to claim 6, wherein the compound is represented by formula 3 or formula 4:

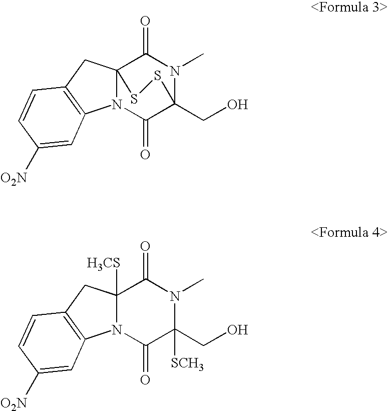

8. The compound according to claim 6, wherein the n is 3 or 4.

9. (canceled)

10. The anticancer agent according to claim 9, wherein the cancer is stomach cancer, liver cancer, colon cancer, lung cancer or prostatic cancer.

11. (canceled)

12. The antibacterial agent according to claim 11, wherein the antibacterial agent shows antibacterial effect on the Sphingomonas sp. bacterial strain deposited under the accession number KCCM10888P, Bacillus subtilis, Proteus vulgaris, Salmonella typhimurium, methicillin resistance Staphylococcus aureus, Aspergillus fumigatus or Trichophyton rubrum.

13. A method for purifying the glionitrin of claim 6 comprising the following steps:
1) Preparing a culture solution by the co-culture method containing the step of culturing the bacterial mixture prepared by adding the Aspergillus sp. fungus strain of claim 2 separately cultured in a liquid medium or the culture solution thereof to the culture solution of the Sphingomonas sp. bacterial strain of claim 1, followed by extracting by adding an organic solvent or an absorption resin to the culture solution;
2) drying the extract of step 1) under reduced pressure, followed by obtaining fractions using column chromatography; and
3) purifying the glionitrin of claim 6 from the fractions of step 2) using column chromatography.

14.-19. (canceled)

20. An anticancer agent containing the culture solution of claim 5 or a compound represented by formula 1 or formula 2: 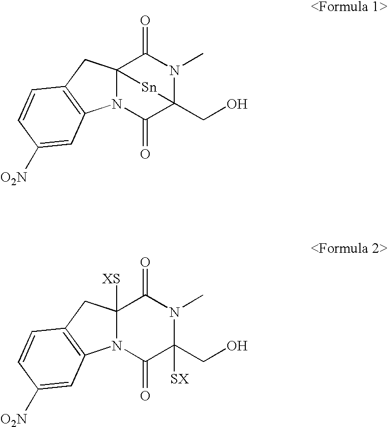
wherein, n is the number of S, which is 1-4; and each X is independently H or alkyl group, and isomers of asymmetric carbons thereof.

21. The anticancer agent of claim 20, wherein the compound is represented by formula 3 or formula 4:

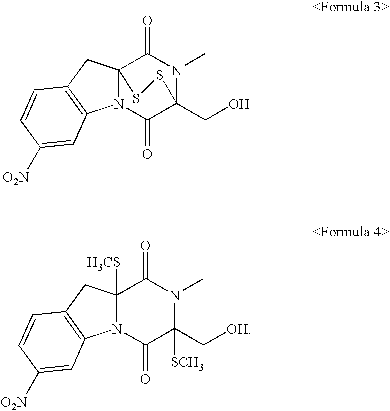

22. An antibacterial agent containing the culture solution of claim 5 or a compound represented by formula 1 or formula 2: 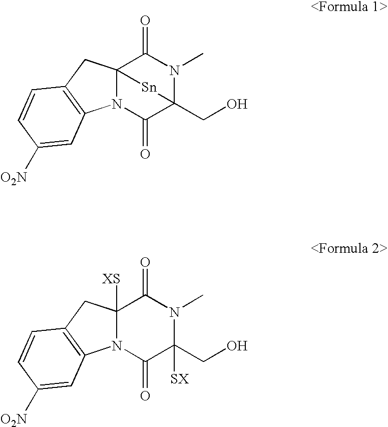
wherein, n is the number of S, which is 1-4; and each X is independently H or alkyl group, and isomers of asymmetric carbons thereof.

23. The antibacterial agent of claim 22, wherein the compound is represented by formula 3 or formula 4:

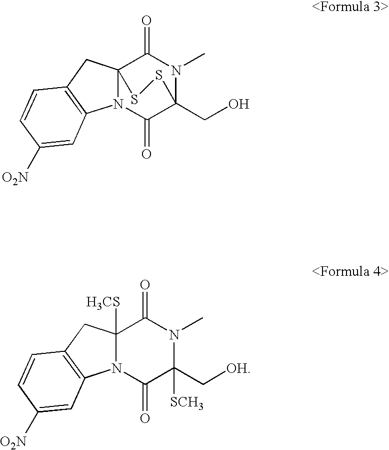

24. A method for the prevention and treatment of cancer containing the step of administering a therapeutically effective dose of the culture solution of claim 5 or a compound represented by formula 1 or formula 2 to a subject in need thereof: 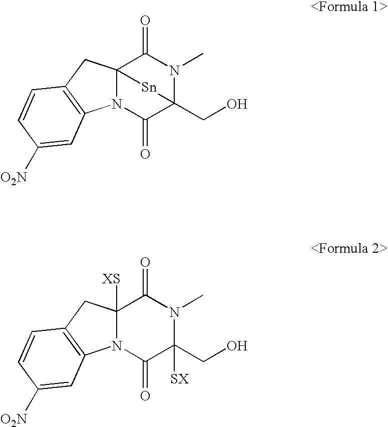
wherein, n is the number of S, which is 1-4; and each X is independently H or alkyl group, and isomers of asymmetric carbons thereof.

25. The method of claim 24, wherein the compound is represented by formula 3 or formula 4:

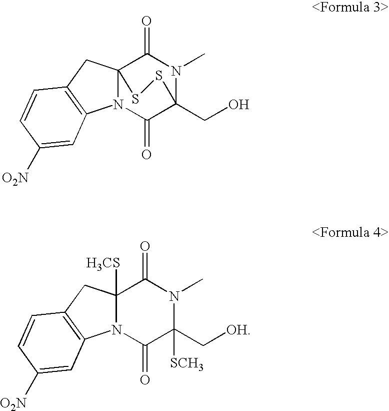

26. A method for the prevention and treatment of bacterial disease containing the step of administering a therapeutically effective dose of the culture solution of claim 5 or a compound represented by formula 1 or formula 2 to a subject in need thereof: 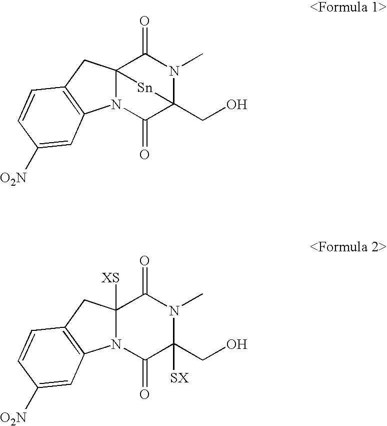
wherein, n is the number of S, which is 1-4; and each X is independently H or alkyl group, and isomers of asymmetric carbons thereof.

27. The method agent of claim 26, wherein the compound is represented by formula 3 or formula 4: [chemical structure]
